(19) United States
(12) Patent Application Publication
(10) Pub. No.: US 2024/0032986 A1
(43) Pub. Date: Feb. 1, 2024

(54) SUCTION ACCESSORY FOR ELECTROCAUTERY SYSTEM

(71) Applicants: Suzanne Lee LOWRY, Atlanta, GA (US); Barry SMITH, Duluth, GA (US)

(72) Inventors: Suzanne Lee LOWRY, Atlanta, GA (US); Barry SMITH, Duluth, GA (US)

(21) Appl. No.: 18/482,957

(22) Filed: Oct. 9, 2023

Related U.S. Application Data
(63) Continuation-in-part of application No. 18/182,524, filed on Mar. 13, 2023.
Application No. 18/182,524 is a continuation of application No. 15/929,485, filed on May 5, 2020, now Pat. No. 11,602,391.
Application No. 15/929,485 is a continuation-in-part of application No. 15/332,957, filed on Oct. 24, 2016, now Pat. No. 10,675,084.
(60) Provisional application No. 63/378,900, filed on Oct. 10, 2022.

(51) Int. Cl. A61B 18/14

(52) U.S. Cl. CPC A61B 18/1402; A61B 2218/008; A61B 2017/00438

(57) ABSTRACT

A cautery pen tip accessory cover comprises a cylindrical member that can receive a cautery pen in a first end and the tip of the cautery pen can extend from a second opposite end when the cover is in a retracted configuration. The cylindrical member has an opened lateral exposure that allows the feel of the cautery pen therethrough. A cover exposure opening in the cylindrical member to provide access to a trigger of the cautery pen when the cautery pen tip cover is in the retracted configuration. The cylindrical member is easily retracted, allowing easy retraction of the cover by the operator squeezing the cover to a resistance bar stabilization system accessory configured over the cautery pen. The cover can include a suction device that removes blood and debris as well as the smoke plume to exist in a single instrument.

### CROSS-REFERENCE TO RELATED APPLICATIONS

This application is a continuation-in-part of U.S. patent application Ser. No. 18/182,524, filed Mar. 13, 2023, which is a continuation of U.S. patent application Ser. No. 15/929,485, filed May 5, 2020, now U.S. Pat. No. 11,602,391, which is a continuation-in-part of U.S. patent application Ser. No. 15/332,957, filed Oct. 24, 2016, now U.S. Pat. No. 10,675,084. This application also claims the benefit of priority to U.S. provisional patent application No. 63/378,900, filed Oct. 7, 2022. The contents of each of the related applications are herein incorporated by reference.

### BACKGROUND OF THE INVENTION

### 1. Field of the Invention

One or more embodiments of the invention relates generally to electrocautery accessories. More particularly, the invention relates to a compression spring automated retractable protective electrocautery pen tip cover.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Many cautery devices alert the physician and staff of their actuation through a high-pitched sound. However, this sound is often not heard secondary to all of the many devices in the operating room which make high pitched sounds.

Currently, the only option to ensure that the pen is not accidentally actuated by either the surgeon, the staff or even the patient, is to place the pen in a large, clumsy plastic box, often referred to as a holster, that is clipped to the operating field. This box is so large that nothing assures that the pen stays in place, other than staff vigilance.

If the pen is dislodged, it can accidently be actuated and could start a fire with the oxygen in the room (for anesthesia) or could burn the patient before someone realizes it is no longer in the holster. Moreover, the holster often comes off of the tether and the opened tip of the pen could be exposed on the operating field. Just leaning on the drapes, for example, can then actuate the opened trigger. Further the pen can be dropped into the field and while under a retractor type instrument, continuous actuation can occur resulting in severe burns or fire.

As early as the turn of the 19th century, discussion of the proper use of suction and its application in the operating room has existed. Suggestions of containing an abscess and its material and not spilling ectopic gestations with meticulous surgical technique were encouraged. Use of syringes and drains were also employed but the suction's usefulness remained in question until 1907. Fear of contamination and trauma to the tissue further delayed acceptance.

In 1907, otolaryngologist Sidney Yankauer revolutionized medical suction and changed the culture of the operating room with what would eventually become the most commonly used medical device for surgery ever invented, the rigid suction catheter. He worked at the outpatient surgery department at Sinai Hospital and while there, invented numerous pieces of medical equipment. He saw the need to clear the patients' airway and gain better visualization during surgical procedures. Made of plastic or metal with small holes in a bulbous, smooth tip and having a comfortable curve such that it is easy to grasp and maneuver made it the perfect solution. Its use in dentistry became the standard then, just as it is today.

As can be seen, there is a need for a protective cautery pen tip cover that protects the pen tip and prevents accidental actuation of the pen trigger while also providing a suction device as part of the electrocautery gun.

### SUMMARY OF THE INVENTION

As medicine and technology have continued to change and improve drastically over the last 200 years, so has the suction tip to include the application of suction to improve visualization, control vessels and contain contaminated spillage. Suction removal of blood and debris are the basic tenant of visualization without which many procedures—whether laparoscopic, open or robotic—would be impossible.

The electrocautery pen because of its typical configuration is a stand-alone device that requires separate suction of the cautery-surgical area. This is usually performed by the surgeon's other hand or requires that the assistant control the suction device. This is necessary because often the surgeon is either retracting, sponging or using a second instrument. This requires that the surgeon's attention is simultaneously directed toward the assistant's suction performance which is both disruptive and inconvenient.

This significant need offers the opportunity for a new suction device that removes blood and debris as well as the smoke plume to exist in a single instrument. This system should cover the existing electro cautery pen. Having a single device that combines all three of these functions, would change electrocautery to a more efficient and safer system and allow more physician or user independence. Such a system is a much-needed necessary accessory.

Embodiments of the present invention provide a suction unit for an electrocautery pen, comprising an attachment portion extending longitudinally along a length of the electrocautery pen when the suction unit is attached to the electrocautery pen, the attachment portion having a first end operable to connect to suction tubing; a suction ring fluidly connected to a second end of the attachment portion, the suction ring fitting about an end of the electrocautery pen; and a suction tip end fluidly connected to the suction ring, the suction tip end operable to drawn suction in a direction away from a electrocautery tip of the electrocautery pen.

Embodiments of the present invention provide an electrocautery system for an electrocautery pen, comprising a suction unit including an attachment portion extending longitudinally along a length of the electrocautery pen, the attachment portion having a first end operable to connect to suction tubing; a suction ring fluidly connected to a second end of the attachment portion, the suction ring fitting about an end of the electrocautery pen; and a suction tip end fluidly connected to the suction ring, the suction tip end operable to drawn suction in a direction away from a electrocautery tip of the electrocautery pen; and a cautery pen safety cover movable between a first position and a second position, the first position housing the electrocautery tip within the cautery pen safety cover, the first position blocking activation of a trigger of the electrocautery pen, the second position permitting the electrocautery tip to extend from a first end of the cautery pen safety cover, and the second position permitting user access to the trigger of the electrocautery pen.

Embodiments of the present invention provide an electrode pen safety system comprising a stabilization system having a track disposed along a longitudinal axis thereof and a connection member operable to secure an electrode pen thereto; a sleeve slidably received on the track, the sleeve having a first end operable to receive the electrode pen therein, and a second opposite end, operable to permit an electrode pen tip to extend therefrom; and a trigger actuation opening formed as a through hole in the sleeve, the trigger actuation opening positioned to permit a user to activate a trigger of the electrode pen when the electrode pen is secured by the connection member of the stabilization system and the sleeve is positioned in a retracted configuration with the electrode pen tip extending beyond the second opposite end of the sleeve, the sleeve preventing access to the trigger when positioned in a non-retracted position, with the sleeve surrounding the electrode pen tip.

Embodiments of the present invention further provide a sleeve for an electrode pen, comprising a first end operable to receive the electrode pen therein; a second opposite end, operable to permit an electrode pen tip to extend therefrom; a trigger actuation opening formed as a through hole in the sleeve, the trigger actuation opening positioned to permit a user to activate a trigger of the electrode pen when the electrode pen is inserted therein in a retracted configuration with the electrode pen tip extending beyond the second opposite end of the sleeve, the sleeve preventing access to the trigger when positioned in a non-retracted position, with the sleeve surrounding the electrode pen tip; and a lateral opening formed as a through hole through the sleeve, the lateral opening disposed on at least one side of the sleeve and permitting direct contact with the electrode pen when the electrode pen is disposed therein.

Embodiments of the present invention also provide an electrocautery system comprising an electrode pen; a stabilization system having a track disposed along a longitudinal axis thereof and a connection member operable to secure the electrode pen thereto; a sleeve slidably received on the track, the sleeve having a first end receiving the electrode pen therein, and a second opposite end, permitting an electrode pen tip to extend therefrom; a trigger actuation opening formed as a through hole in the sleeve, the trigger actuation opening positioned to permit a user to activate a trigger of the electrode pen when the sleeve is positioned in a retracted configuration with the electrode pen tip extending beyond the second opposite end of the sleeve, the sleeve preventing access to the trigger when positioned in a non-retracted position, with the sleeve surrounding the electrode pen tip; and a compression spring disposed within the sleeve adjacent the second opposite end, the compression spring urging the sleeve to a non-retracted position.

In one aspect of the present invention, a cautery pen tip cover comprises a cylindrical member with opened exposure laterally allowing the feel of the exposed pen operable to receive a cautery pen in a first end thereof; a tip opening disposed in a second, opposite end of the cylindrical member, the tip opening permitting a tip of the cautery pen to extend therethrough when the cautery pen tip cover is in a retracted configuration; a cover exposure opening formed in the cylindrical member, the cover exposure opening providing access to a trigger of the cautery pen when the cautery pen tip cover is in the retracted configuration, wherein the cautery pen tip cover prevents access to the trigger when the cautery pen tip cover is in a non-retracted configuration; and a resilient member operable to urge the cautery pen tip cover to the non-retracted configuration. The cylindrical member is easily retracted by a finger ring with a raised surface configured permanently into the cylindrical member allowing easy retraction in a natural closed hand position of the operator squeezed to a resistance bar stabilization system accessory configured over the body of the pen.

In another aspect of the present invention, a cautery pen tip cover comprises a cylindrical member with opened exposure laterally allowing the feel of the exposed pen operable to receive a cautery pen in a first end thereof; a tip opening disposed in a second, opposite end of the cylindrical member, the tip opening permitting a tip of the cautery pen to extend therethrough when the cautery pen tip cover is in a retracted configuration by squeezing the finger rings against the resistance bar accessory in a natural closed hand position; a cover exposure opening formed in the cylindrical member, the cover exposure opening providing access to a trigger of the cautery pen when the cautery pen tip cover is in the retracted configuration, wherein the cautery pen tip cover prevents access to the trigger when the cautery pen tip cover is in a non-retracted configuration; a compression spring operable to urge the cautery pen tip cover to the non-retracted configuration; the one or more cover notch guides formed on an inside surface of the cylindrical member identically fits into the notch guides formed in the stabilization system, which consists of the tracks, the resistance bar and the receptacle for an optional hollow tube suction system attached to the cautery pen; and at least one cover finger plate extending from the cylindrical member at the first end thereof.

In a further aspect of the present invention, a cautery system comprises a cautery pen; and a cautery pen tip cover with exposed lateral openings, the cautery pen tip cover comprises a cylindrical member operable to receive the cautery pen in a first end thereof; a tip opening disposed in a second, opposite end of the cylindrical member, the tip opening permitting a tip of the cautery pen to extend therethrough when the cautery pen tip cover is in a retracted configuration; a cover exposure opening formed in the cylindrical member, the cover exposure opening providing access to a trigger of the cautery pen when the cautery pen tip cover is in the retracted configuration, wherein the cautery pen tip cover prevents access to the trigger when the cautery pen tip cover is in a non-retracted configuration; and a resilient member operable to urge the cautery pen tip cover to the non-retracted configuration.

In a further aspect of the present invention, the cautery system is simplified and comprises a stabilization system including a resistance bar, a track with an optional suction-tubing reservoir that is attached in one piece by a wrapped configuration on one end and spot welding on the opposite end to allow flawless retraction of the cautery pen tip/trigger cover; a two part system which does not alter the original pen but is a simple accessory system with a resilient member which urges the cautery cover in the non-retracted position.

In another aspect of the present invention, a method for preventing inadvertent activation of an electrode pen comprises mounting the electrode pen to a connection member of a stabilization system having a track disposed along at least a portion of a longitudinal axis thereof; slidably receiving a sleeve on the track, the sleeve having a first end receiving the electrode pen therein, and a second opposite end, permitting an electrode pen tip to extend therefrom; and sliding the sleeve between a retracted position, where a trigger actuation opening in the sleeve permits a user to activate a trigger of the electrode pen and the electrode pen tip extends beyond the second opposite end of the sleeve, and a non-retracted position, where the sleeve prevents access to the trigger.

In a further aspect of the present invention, a method for preventing inadvertent activation of an electrode pen comprises mounting the electrode pen to a connection member of a stabilization system having a track disposed along at least a portion of a longitudinal axis thereof; slidably receiving a sleeve on the track, the sleeve having a first end receiving the electrode pen therein, and a second opposite end, permitting an electrode pen tip to extend therefrom; sliding the sleeve between a retracted position, where a trigger actuation opening in the sleeve permits a user to activate a trigger of the electrode pen and the electrode pen tip extends beyond the second opposite end of the sleeve, and a non-retracted position, where the sleeve prevents access to the trigger; covering the electrode pen tip with the sleeve in the non-retracted position; and urging the sleeve into the non-retracted position when an absent of external force is present on the sleeve.

In another aspect of the present invention, a method for automatically preventing inadvertent activation of an electrode pen comprises mounting the electrode pen to a connection member of a stabilization system having a track disposed along at least a portion of a longitudinal axis thereof; slidably receiving a sleeve on the track, the sleeve having a first end receiving the electrode pen therein, and a second opposite end, permitting an electrode pen tip to extend therefrom; sliding the sleeve between a retracted position, where a trigger actuation opening in the sleeve permits a user to activate a trigger of the electrode pen and the electrode pen tip extends beyond the second opposite end of the sleeve, and a non-retracted position, where the sleeve prevents access to the trigger; covering the electrode pen tip with the sleeve in the non-retracted position; and automatically moving the sleeve into the non-retracted position from the retracted position when a user removes a user-applied force to the sleeve.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a perspective view of a cautery pen accessory cover mounted on a stabilization bar system with the pen disposed therein in an actuated position, according to an exemplary embodiment of the present invention;

FIG. 2 illustrates a perspective view of the stabilization bar system of FIG. 1, separate from the pen, showing a track, with guide notches and an optional tubing track, for receiving the cautery pen accessory cover according to an exemplary embodiment of the present invention;

FIG. 3 illustrates a side view of the cautery pen accessory cover, partially cut away to show a spring therewithin, according to an exemplary embodiment of the present invention;

FIG. 4 illustrates a left side view of the cautery pen accessory cover/stabilization bar system of FIG. 1;

FIG. 5 illustrates a right side view of the cautery pen accessory cover/stabilization bar system of FIG. 1;

FIG. 6 illustrates a perspective view of the cautery pen accessory cover/stabilization bar system of FIG. 1, showing the cautery pen in a non-retracted, protected position;

FIG. 7 illustrates a perspective view of a cautery pen accessory cover according to an alternate embodiment of the present invention;

FIG. 8 illustrates a perspective view of a cautery pen accessory cover according to an alternate embodiment of the present invention;

FIG. 9 illustrates a detailed perspective view showing use of the cautery pen accessory cover of FIG. 8;

FIG. 10 illustrates an exploded perspective view of the cautery pen safety system of FIG. 1;

FIG. 11 illustrates a perspective view of a cautery pen disposed in the stabilization bar system without the accessory cover installed thereupon;

FIG. 12 illustrates a perspective view of a cautery pen cover disposed on a cautery pen according to another embodiment of the present invention;

FIG. 13 illustrates an exploded view of the cautery pen cover of FIG. 12;

FIG. 14 illustrates a perspective view of a first embodiment of a cautery pen cover, in a safety position on a cautery pen, with a suction catheter attached thereto, according to an exemplary embodiment of the present invention;

FIG. 15 illustrates a perspective view of a second embodiment of a cautery pen cover, in a safety position on a cautery pen, with a suction catheter attached thereto, according to an exemplary embodiment of the present invention;

FIG. 16 illustrates a perspective view of the cautery pen cover of FIG. 14 in the cautery pen use position;

FIG. 17 illustrates a perspective view of the cautery pen cover of FIG. 15 in the cautery pen use position;

FIG. 18 illustrates an exploded perspective view of the cautery pen cover of FIG. 14;

FIG. 19 illustrates an exploded perspective view of the cautery pen cover of FIG. 15; and

FIG. 20 illustrates a perspective view of the suction attachment used with the cautery pen covers of FIGS. 14 and 15.

The illustrations in the figures may not necessarily be drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a cautery pen tip accessory cover that comprises a cylindrical member that can receive a cautery pen in a first end and the tip of the cautery pen can extend from a second opposite end when the cover is in a retracted configuration. The cylindrical member has an opened lateral exposure that allows the feel of the cautery pen therethrough. A cover exposure opening, also referred to as a trigger actuation opening, in the cylindrical member to provide access to a trigger of the cautery pen when the cautery pen tip cover is in the retracted configuration. A resilient member urges the cautery pen tip cover to the non-retracted configuration where access to the cautery pen trigger is prevented. The cylindrical member is easily retracted by a finger ring configured permanently into the cylindrical member, allowing easy retraction of the cover by the operator squeezing the cover to a resistance bar stabilization system accessory configured over the cautery pen. The cautery pen tip accessory cover can include a suction device that may be used to remove debris and smoke away from the surgical field.

The simplicity of the cautery system is that the cautery pen does not have to be altered, other than the addition of injection molded plastic attachments, to achieve the final product. The compression spring specifications can be designed to provide appropriate retraction of the cautery pen tip into the cover when the pen is not maintained in the retracted configuration. There is simply no other mechanism that assures coverage of the electrode tip other than a permanently attached sleeve that forces compliance of the operator by removing the option of utilizing a holster that is clumsy and remote from the device. This sleeve requires retraction to actuate the device and remains covered in the non-use state. This reduces accidental actuation, burns and operating room fires by covering the source for ignition.

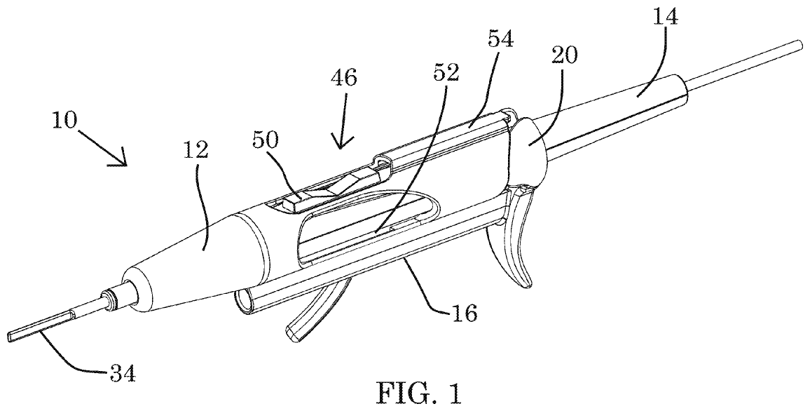

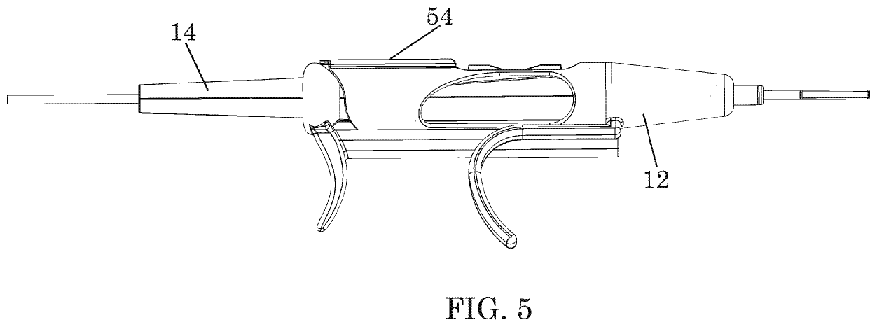

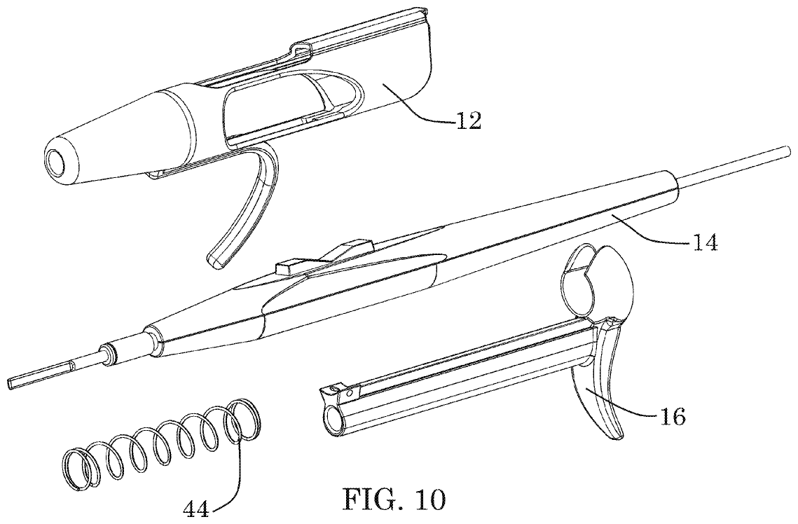

Referring now to FIGS. 1 through 5 and 10, a cautery pen safety system 10, also referred to as safety system 10 or simply, system 10, can include an accessory sleeve 12 that can cover the tip of an electrode pen 14, also referred to as a cautery pen 14. The sleeve 12 can be easily added to a conventional electrode pen. The sleeve 12 can move along a track 18 of a stabilization system 16 while the electrode pen 14 is fixed to a pen wrap portion 20 of the stabilization system 16.

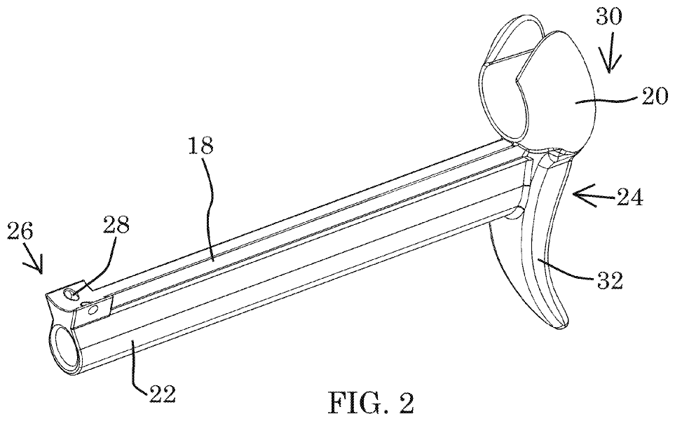

Referring specifically to FIG. 2, the stabilization system 16 can include the track 18 extending longitudinally along a length of the stabilization system 16. A suction tube 22 may be disposed below or adjacent the track 18. In use, suction may be applied to a suction tube distal end 24 to create, for example, a built-in smoke evacuator for use during cauterization.

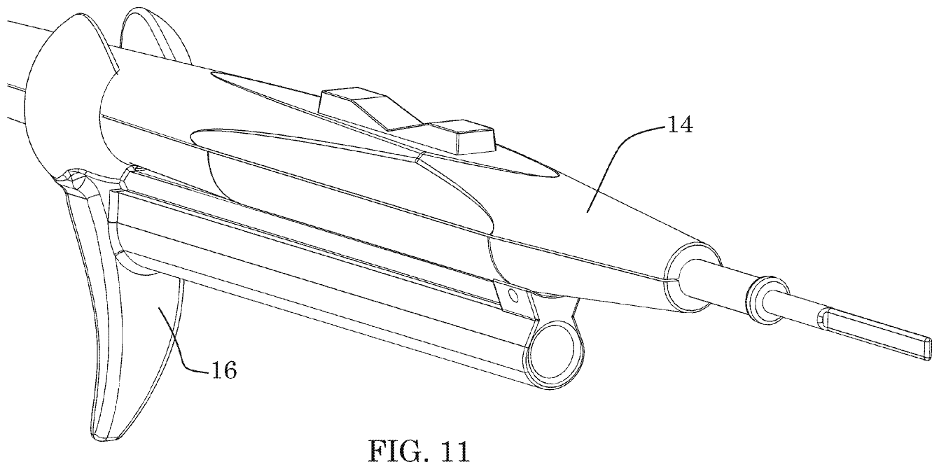

A proximate end 26 of the stabilization system 16 can include one or more spot welds 28, or similar protrusions can allow direct attachment of the electrode pen 14 to the stabilization system 16. A distal end 30 of the stabilization system 16 can include the pen wrap portion 20 which can be configured to secure the electrode pen 14 therein, as shown in FIGS. 1 and 11. The pen wrap portion 20 may be a friction fitting member, or may include one or more securement mechanisms (not shown) for fixing the electrode pen 14 thereto. The securement mechanisms may include set screws, tightening members, or the like. In some embodiments, the electrode pen 14 may be made integrally with the stabilization system 16. In this embodiment, the pen wrap portion 20 may not be necessary since the pen 14 is already affixed to the integral stabilization system.

A resistance bar 32 may extend, at or near the distal end 30, away from the pen wrap portion 20 to provide a fixed extension effectively attached to the electrode pen 14 that assists in the use and retraction of a tip 34 of the electrode pen 14 from the sleeve 12.

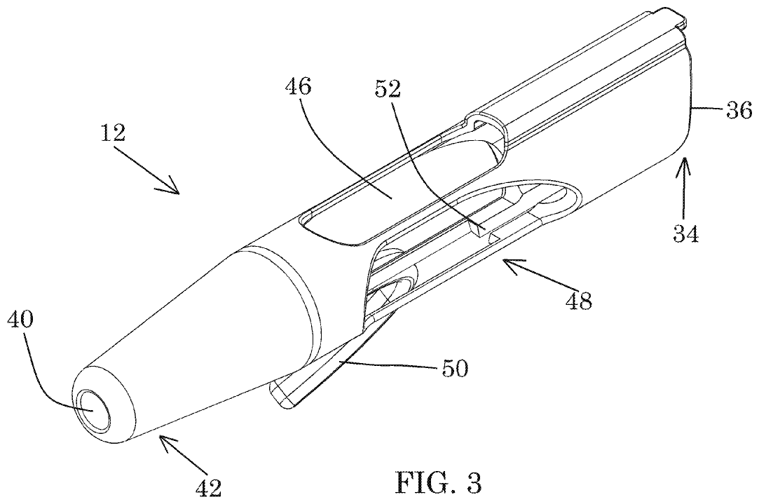

Referring now to FIGS. 3 through 5, the sleeve 12 can include a first opening 36 at a sleeve distal end 38 into which the electrode pen 14 can fit. A tip opening 40 can be formed in a sleeve proximate end 42 of the sleeve 12. A compression spring 44 may be disposed inside the sleeve 12, at the sleeve proximate end 42. The compression spring 44 may be sized too large to extend out of the sleeve proximate end 42 but may be small enough to press against an electrode pen body tip end 46, from which the electrode tip 34 extends. The effect is that the sleeve 12 may be disposed over the electrode pen 14 and, as the sleeve 12 is retracted to expose the electrode pen tip 34 out from the tip opening 40 of the sleeve 12, the compression spring 44 urges the sleeve 12 to move in an opposite direction to cover the electrode pen tip 34. The compression spring 44 may be fixed inside of the sleeve 44 or may simply be removably disposed therein.

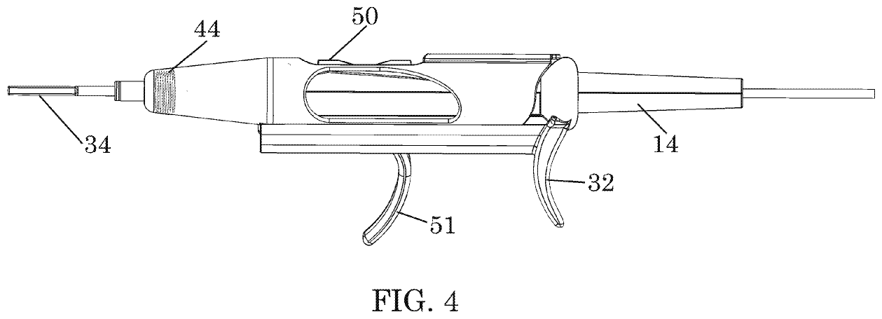

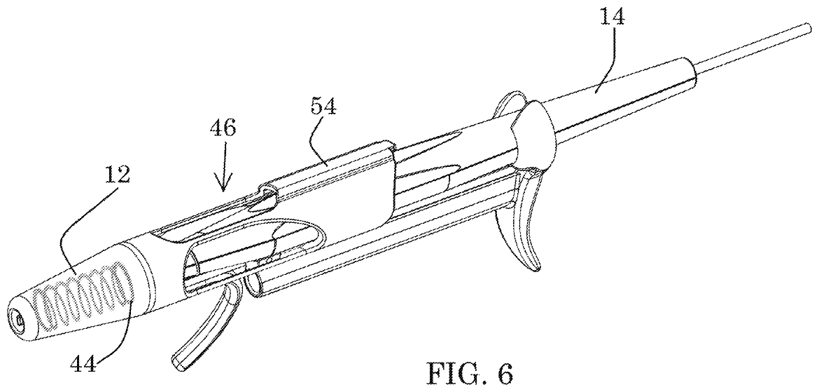

The sleeve 12 can include a trigger actuation opening 46 formed as a hole through a portion of the sleeve 12. The trigger actuation opening 46 is positioned such that when the sleeve 12 is in a non-retracted position (see FIG. 6), the sleeve 12 prevents access to a trigger 50 of the electrode pen 14. However, when the sleeve 12 is moved, against the resiliency of the compression spring 44, into the retracted configuration (as shown in FIGS. 1 and 4, for example), the electrode pen tip 34 extends from the tip opening 40 and the trigger 50 is accessible through the trigger actuation opening 46. Should the unit be dropped or set aside, the resiliency of the compression spring 44 can cause the sleeve to slide along the track 18 (see FIG. 2) of the stabilization system 16 into the non-retracted configuration, where the electrode pen tip 34 is protected inside the sleeve 12 and the trigger 50 is non-accessible, covered by the sleeve 12.

The sleeve 12 can include a lateral opening 48 along at least a portion of the side of the sleeve 12. The lateral opening 48 can be formed on one or both sides of the sleeve 12 and allows the user direct contact with the electrode pen 14 disposed in the sleeve 12. Typically, the lateral opening 48 is disposed between the trigger actuation opening 46 and the bottom of the sleeve 12, where the sleeve 12 rides along the track 18 of the stabilization system 16. In some embodiments, the lateral opening 48 may be disposed to be longer or shorter than a length of the trigger actuation opening 46.

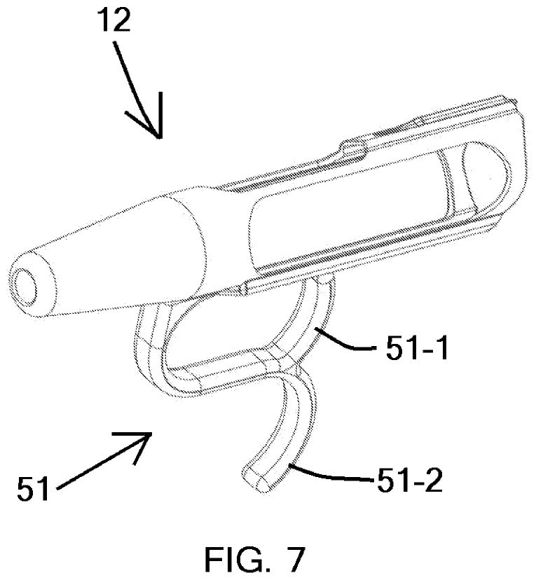

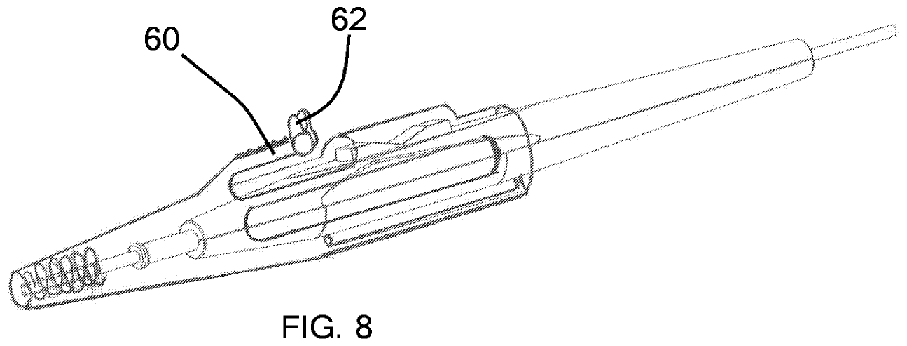

The sleeve 12 can include a finger ring 51 affixed to a portion of the sleeve 12 and extending downward (toward the stabilization system 16 when the system 10 is assembled, as shown in FIG. 4, for example). The finger ring 51 may be formed as a curved bar, as shown in FIG. 4, or may have other shapes, such as a ring shape as shown in FIG. 7, or may be excluded, as shown in FIG. 8, for example. The finger ring 51 may be used to assist the user in retracting the sleeve 12 by, for example, allowing the user to squeeze the finger ring 51 toward the resistance bar 32 to achieve the retracted configuration of FIG. 4.

When not in use, the operator releases the sleeve 12 and the compression spring 44 recoils, covering the electrode pen tip 34 and is stopped by a graduated ring or trigger lever (not shown), or other like mechanism, that prevents the base of the sleeve 12 from propelling completely off of the electrode pen 14 and stops at the point that the electrode pen tip 34 is completely covered.

Referring now to FIGS. 1 and 6, one can see how the sleeve 12 can be moved from the non-retracted position as shown in FIG. 6, where the trigger actuation opening 46 does not allow activation of the trigger 50 (not shown, but covered by a portion 54 of the sleeve 12), to the retracted position as shown in FIG. 1, where the electrode pen tip 34 extends from the sleeve 12 and the trigger actuation opening 46 allows a user to directly contact the trigger 50 of the electrode pen 14. It should be noted that, in some embodiments, the portion 54 of the sleeve 12 that covers the trigger 50 of the electrode pen 14 may be raised, as shown in FIG. 6, thereby allowing the electrode pen 14 to move linearly through the sleeve 12.

The sleeve 12 may include a channel 52 or a set of grooves or protrusions formed along the bottom thereof to receive the track 18 of the stabilization system 16. This configuration allows the sleeve 12 to slide along the track 18 and permits the movement between the retracted (use) and non-retracted (stored) positions.

Referring to FIG. 7, in some embodiments, the sleeve 12 may be formed from a varied design. For example, the finger ring 51 may include a ring portion 51-1 and an extending portion 51-2. Of course, other configurations are contemplated within the scope of the present invention.

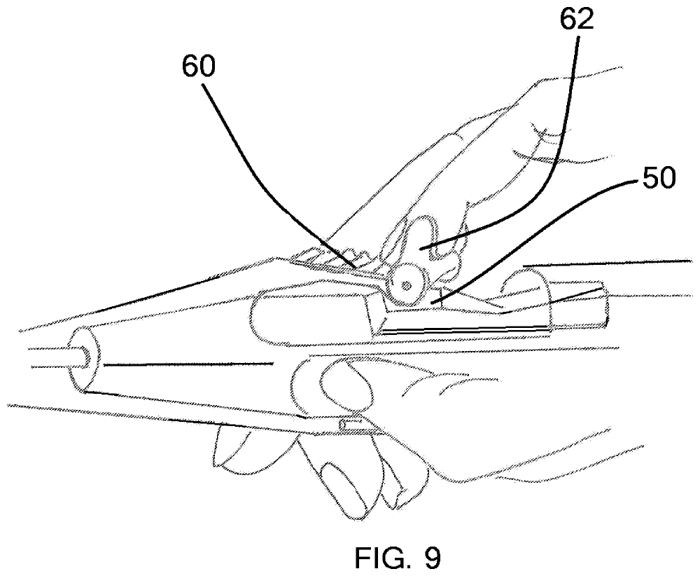

Referring to FIGS. 8 and 9, in some embodiments, the sleeve 12 can include a flexible actuation member 60 that may extend over the trigger actuation opening 46 as shown in FIG. 9. The actuation member 60 may include a textured outer surface for receiving a user's finger. When the system is in the retracted configuration, the trigger 50 may be disposed below the actuation member 60, which the user may flexibly depress against the trigger 50 to actuate the electrode pen 14. In some embodiments, finger stabilization side members 62 may be disposed on opposite sides of the flexible actuation member 60.

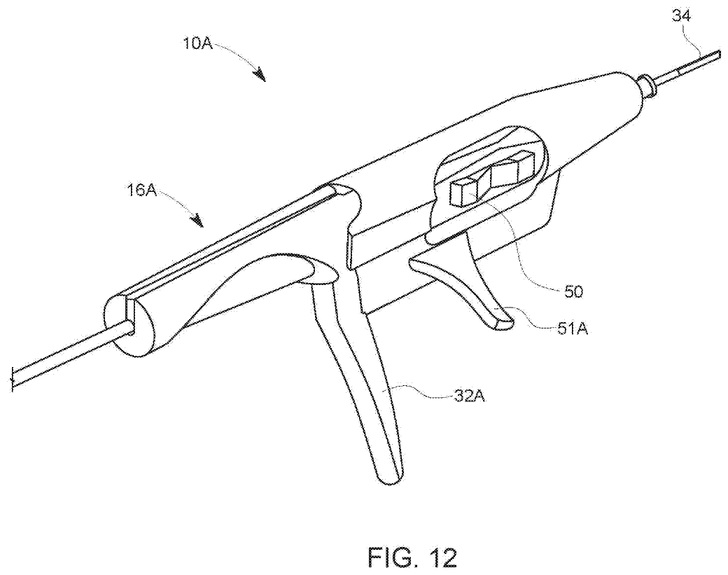

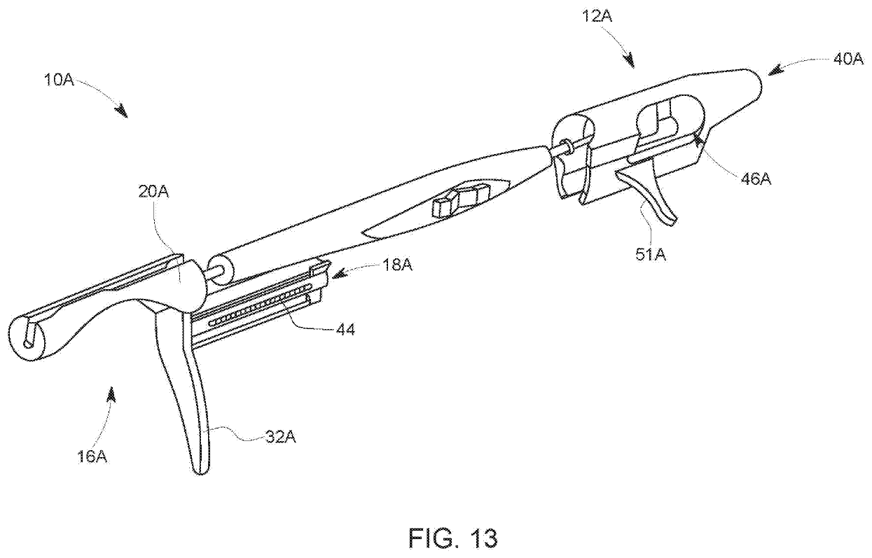

Referring now to FIGS. 12 and 13, a cautery pen system 10A can include a stabilization system 16A for holding the cautery pen 14. A pen wrap portion 20A of the stabilization system 16A may retain a base of the cautery pen 14. A sleeve 12A may slide on a track 18A of the stabilization system 16A. When a trigger 51A of the sleeve 12A is pulled toward a resistance bar 32A of the stabilization system 16A, the electrode tip 34 of the cautery pen 14 may extend from a tip opening 40A of the sleeve 12A. A spring 44A may be disposed in the stabilization system 16A, typically running alongside the track 18A. When an external force (such as a user's fingers) is used to pull the sleeve 12A to extend the electrode tip 34, the spring 44A may be resiliently compressed. When this external force is removed (such as when the cautery pen, in the cautery pen system, is placed on a stand, is dropped, or the like), the spring 44A causes the sleeve 12A to cover the electrode tip 34 of the cautery pen 14.

The embodiment of FIGS. 12 and 13 is similar to those described above with the following additional benefits. (1) The pen has been rotated to the side such that the operator's index finger is opposite the thumb for better control and a naked pen feel; (2) the stabilization piece has been extended such that the operator's hand is fitted for greater control and resistance; (3) the retraction arm is sleek with no loops and the angle is more in line with the shape of the hand; (4) the base piece now securely curls around the pen with a secure snap on placement requiring no other measures to attach to the naked pen; and (5) the spring has been placed into the base piece prior to snapping on allowing easy two piece assembly over any existing pen with little modification.

As discussed above, the cautery pen cover system (also referred to as the C-PAS system) is a spring actuated device that encloses an existing cautery pen while covering the hot tip and the rocker trigger in the closed position, as described in U.S. Pat. Nos. 9,474,572 and 10,675,084 10, herein incorporated by reference. This system has endless safety features and allows for other major secondary accessories such as a clip-on plume removal attachment, such as suction tube 22, discussed above. These accessory systems do not alter the function of the cautery pen but enhance the device and allow other needed capabilities of the C-PAS system currently not available.

As described in greater detail below, the C-PAS system also allows that a debris suction system can be snapped over the tip with rigid tubing which neatly attaches to the existing retracting safety cover. By increasing the tubing size and pneumatic pressure (remote from the surgical field and currently the standard for Yankauer systems), the “plume remover system” after much alteration, becomes a wall-based or battery driven standard suction device for plume, blood and debris removal, all in one. Debris and plume are simultaneously removed allowing the surgeon both complete suction control and enhanced visualization, without the need of a separate technician operating the suction device. By retracting the cover and suction tip, exposure of the electrocautery tip and trigger simultaneously allows routine use of the cautery while slowly blocking the tubing and pneumatic pressure. This allows the physician to control suction volume as well as determining when and where it is used.

The advantages of having a clear, clean, suctioned surgical field provided by a single device, has endless advantages over a separate suction device. The surgeon is free to choose when and where to apply suction or to use only the cautery system while never involving an assistant unless requested. The suction system can selectively be removed without requiring a new system by detaching the suction accessory from the C-PAS system. No other cautery device has this option.

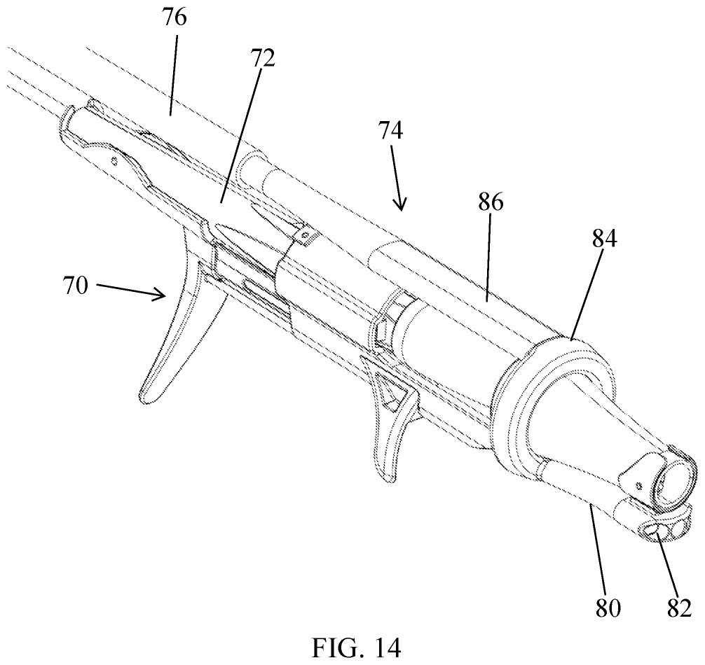

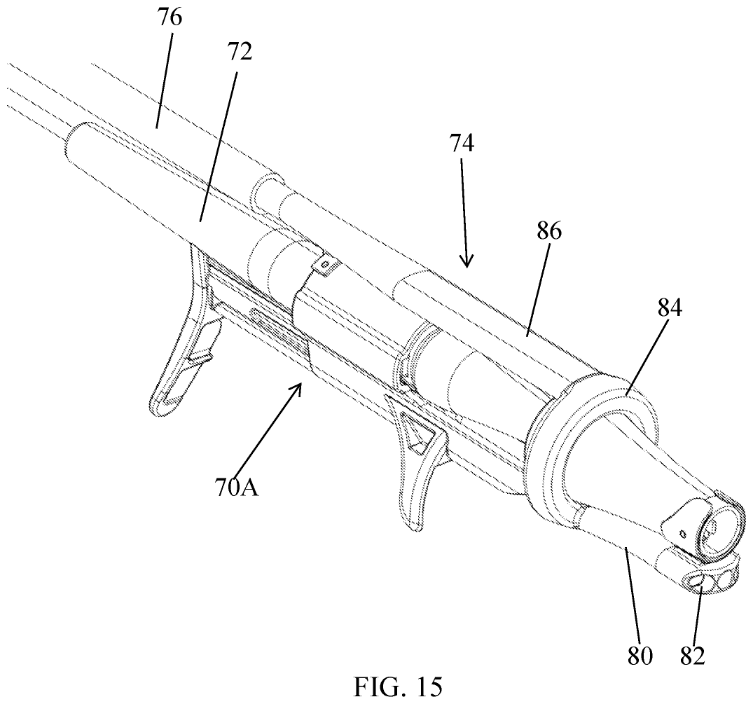

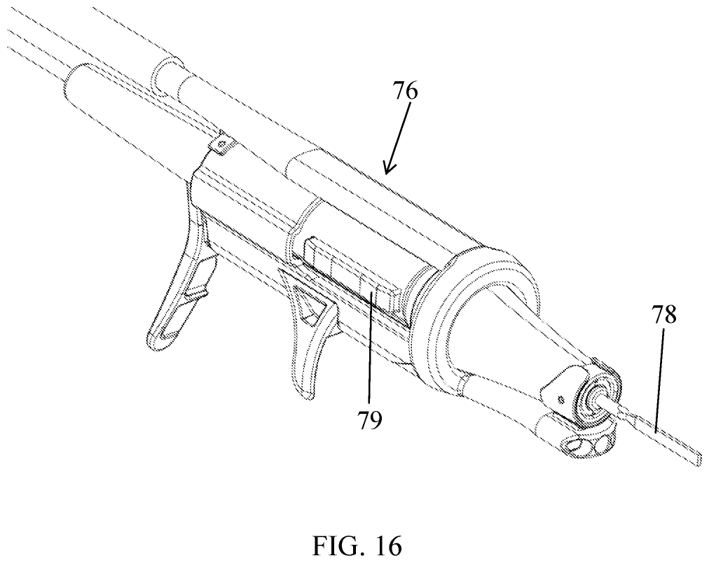

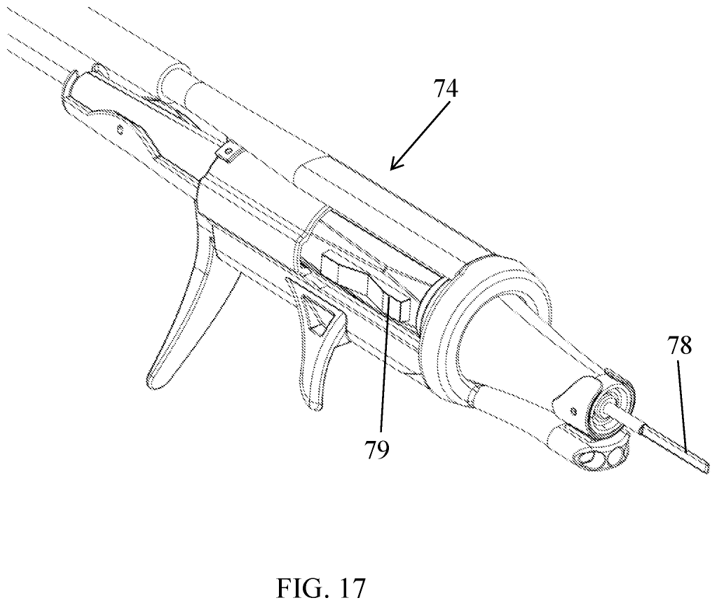

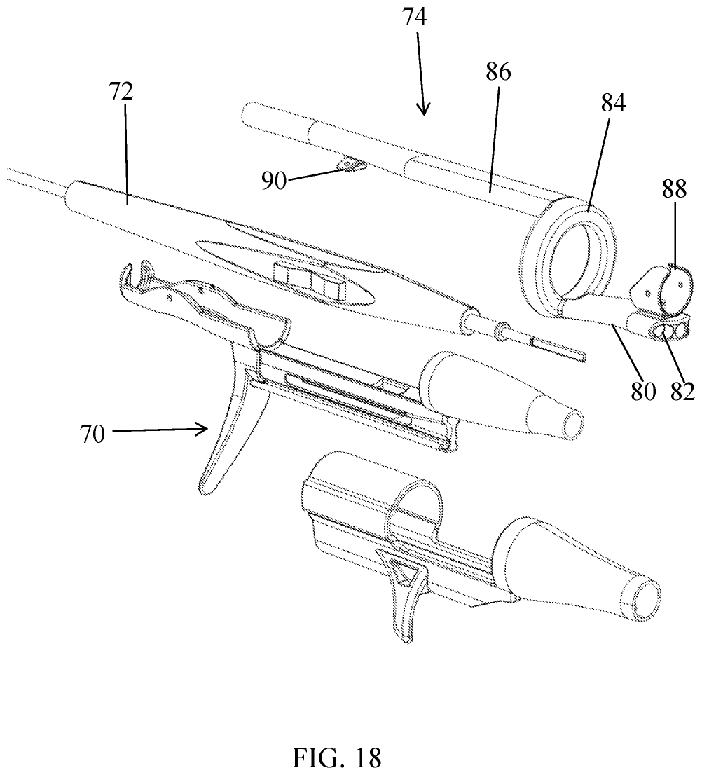

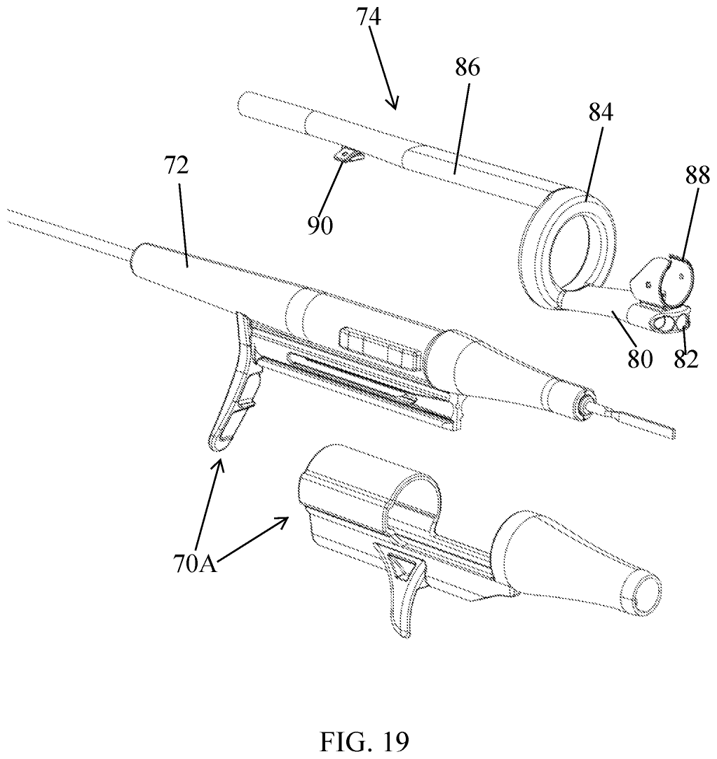

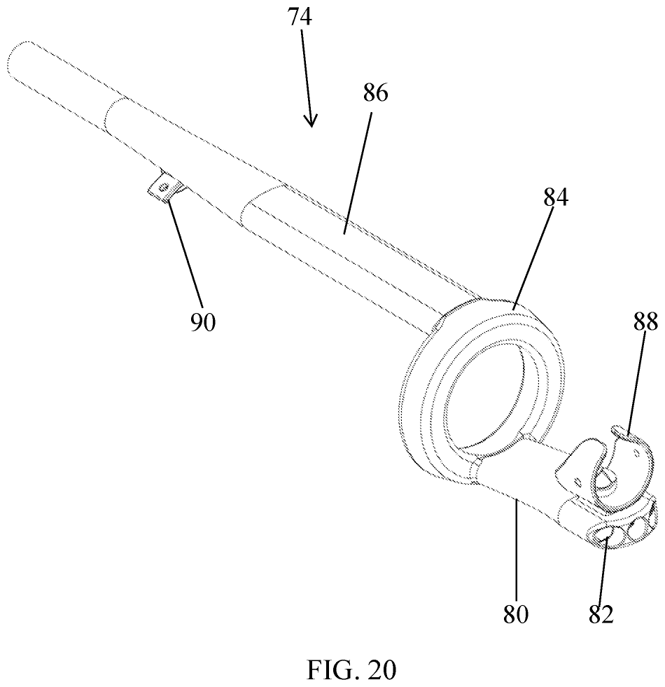

Referring now to FIGS. 14 through 20, a cautery pen 72 can be used with a cautery pen safety cover 70, 70A, where, as discussed in greater detail above, the cautery pen safety cover 70, 70A can cover a cautery pen trigger 79 and keep the cautery pen tip 78 within the safety cover 70, 70A unless a user pulls a trigger, thus exposing the cautery pen trigger 79 and the cautery pen tip 78. As discussed above, this system provides tremendous advantages to the field of electrocautery.

According to aspects of the present invention, a rigid suction catheter 74, similar in size to a Yankauer catheter, can be attached to the safety cover 70, 70A. For example, a suction ring 84 can fit about a tip of the safety cover 70, 70A, as illustrated. A clip 88 can also clip to the end of the tip of the safety cover 70, 70A to retain the catheter 74 in position on the safety cover 70, 70A. A mount 90 may also be provided to further secure the catheter 74 in position. A standard high volume suction line 76, such as those used with in-room or portable suction units, can be attached to a longitudinal attachment portion 86 of the catheter 74.

When suction is applied, the suction can draw through the longitudinal attachment portion 86, which is fluidly connected to the suction ring 84. In some embodiments, the suction ring 84 is formed as hollow, tubular member. An extension portion 80 can fluidly connect the suction ring 84 with a suction tip end 82 of the catheter 74. The length of the extension portion 80 can align the suction tip end 82 with the end of the safety cover 70, 70A, as illustrated. In some embodiments, the suction tip end 82 may be formed from a plurality of openings or may be a single large opening.

A suction control hole (not shown) may be formed in the catheter 74, where a user can place their finger over the hole to cause an increase in suction at the suction tip end 82. Of course, the catheter 74 may function with or without such a suction control hole.

Further, while the catheter 74 is shown attached to the tip of the safety cover 70, 70A, in some embodiments, the catheter 74 may attach directly to the electrocautery pen 72. Such an embodiment may be useful when the safety cover 70, 70A is not used with the cautery pen.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A suction unit for an electrocautery pen, comprising:
an attachment portion extending longitudinally along a length of the electrocautery pen when the suction unit is attached to the electrocautery pen, the attachment portion having a first end operable to connect to suction tubing;
a suction ring fluidly connected to a second end of the attachment portion, the suction ring fitting about an end of the electrocautery pen; and
a suction tip end fluidly connected to the suction ring, the suction tip end operable to drawn suction in a direction away from a electrocautery tip of the electrocautery pen.

2. The suction unit of claim 1, further comprising an extension portion fluidly extending from the suction ring to position the suction tip end adjacent the electrocautery tip.

3. The suction unit of claim 1, further comprising a cautery pen safety cover movable between a first position and a second position, the first position housing the electrocautery tip within the cautery pen safety cover, the first position blocking activation of a trigger of the electrocautery pen, the second position permitting the electrocautery tip to extend from a first end of the cautery pen safety cover, and the second position permitting user access to the trigger of the electrocautery pen.

4. The suction unit of claim 3, wherein the suction ring fits about the cautery pen safety cover, adjacent the first end thereof.

5. The suction unit of claim 3, further comprising a clip operable to fix the suction unit to the cautery pen safety cover.

6. The suction unit of claim 3, wherein the suction tip end is positioned directly adjacent the first end of the cautery pen safety cover.

7. The suction unit of claim 1, wherein the suction unit is formed entirely from a rigid material.

8. The suction unit of claim 1, wherein the suction ring is a hollow tubular member.

9. An electrocautery system for an electrocautery pen, comprising:
a suction unit including:
an attachment portion extending longitudinally along a length of the electrocautery pen, the attachment portion having a first end operable to connect to suction tubing;
a suction ring fluidly connected to a second end of the attachment portion, the suction ring fitting about an end of the electrocautery pen; and
a suction tip end fluidly connected to the suction ring, the suction tip end operable to drawn suction in a direction away from a electrocautery tip of the electrocautery pen; and
a cautery pen safety cover movable between a first position and a second position, the first position housing the electrocautery tip within the cautery pen safety cover, the first position blocking activation of a trigger of the electrocautery pen, the second position permitting the electrocautery tip to extend from a first end of the cautery pen safety cover, and the second position permitting user access to the trigger of the electrocautery pen.

10. The electrocautery system of claim 9, wherein the suction unit further includes an extension portion fluidly extending from the suction ring to position the suction tip end adjacent the electrocautery tip.

11. The electrocautery system of claim 10, wherein a length of the extension portion causes the suction tip end to be positioned directly adjacent the first end of the cautery pen safety cover.

12. The electrocautery system of claim 9, wherein the suction ring fits about the cautery pen safety cover, adjacent the first end thereof.

13. The electrocautery system of claim 9, further comprising a clip operable to fix the suction unit to the cautery pen safety cover.
